(19) United States
(12) Patent Application Publication
(10) Pub. No.: US 2023/0210576 A1
(43) Pub. Date: Jul. 6, 2023

(54) ELECTROSURGICAL APPARATUS

(71) Applicant: Creo Medical Limited, Chepstow (GB)

(72) Inventors: John Bishop, Chepstow (GB); Chris Hancock, Chepstow (GB)

(73) Assignee: Creo Medical Limited, Chepstow (GB)

(21) Appl. No.: 17/927,794

(22) PCT Filed: Jun. 2, 2021
(86) PCT No.: PCT/EP2021/064860

§ 371 Date: Nov. 25, 2022

(30) Foreign Application Priority Data
Jun. 5, 2020 (GB) 2008464.6

(51) Int. Cl. A61B 18/12; A61B 18/14; A61B 18/18; H02J 50/12

(52) U.S. Cl. CPC A61B 18/1206; A61B 18/14; A61B 18/1815; H02J 50/12; A61B 2018/1226; A61B 2018/00601

(57) ABSTRACT

An electrosurgical apparatus is provided having a rechargeable power source which may be charged wirelessly. The apparatus comprises an oscillator for generating electromagnetic (EM) energy (e.g. radiofrequency energy or microwave frequency energy); a controller operable to select an energy delivery profile for the oscillator; a feed structure for conveying the electromagnetic energy to an output; a rechargeable power source arranged to supply power to the oscillator; and a receiver circuit comprising an inductive coupler configured to wirelessly receive power from a transmitter and supply received power to the rechargeable power source. The selection of an energy delivery profile may involve switching the oscillator on or off in one example, or may comprise more complex operation such as the selection of a pulse profile in some embodiments.

### FIELD OF THE INVENTION

The invention relates to an electrosurgical apparatus for generating radiofrequency and/or microwave frequency electromagnetic energy which may be used to treat biological tissue. In particular, the invention relates to an electrosurgical apparatus having a rechargeable power source which may be charged wirelessly. In some embodiments, the rechargeable power source is configured for wired charging.

### BACKGROUND TO THE INVENTION

Electrosurgery utilises radiofrequency (RF) and/or microwave frequency electromagnetic (EM) energy to treat biological tissue, for example by using the RF and/or microwave EM energy to cut and/or coagulate tissue. Typically, electrosurgery requires the use of large generators to provide the RF and/or microwave EM energy. However, advances in solid state technology mean that smaller generators are now possible and these generators may be transportable.

GB 2 486 343 discloses a control system for an electrosurgical apparatus which delivers both RF and microwave energy to treat biological tissue. The energy delivery profile of both RF energy and microwave energy delivered to a probe is set based on sampled voltage and current information of RF energy conveyed to the probe and sampled forward and reflected power information for the microwave energy conveyed to and from the probe.

FIG. 1 shows a schematic diagram of an electrosurgical apparatus 400 as set out in GB 2 486 343. The apparatus comprises a RF channel and a microwave channel. The RF channel contains components for generating and controlling an RF frequency electromagnetic signal at a power level suitable for treating (e.g. cutting or desiccating) biological tissue. The microwave channel contains components for generating and controlling a microwave frequency electromagnetic signal at a power level suitable for treating (e.g. coagulating or ablating) biological tissue.

The microwave channel has a microwave frequency source 402 followed by a power splitter 424 (e.g. a 3 dB power splitter), which divides the signal from the source 402 into two branches. One branch from the power splitter 424 forms a microwave channel, which has a power control module comprising a variable attenuator 404 controlled by controller 406 via control signal V₁₀ and a signal modulator 408 controlled by controller 406 via control signal V₁₁, and an amplifier module comprising drive amplifier 410 and power amplifier 412 for generating forward microwave EM radiation for delivery from a probe 420 at a power level suitable for treatment. After the amplifier module, the microwave channel continues with a microwave signal coupling module (which forms part of a microwave signal detector) comprising a circulator 416 connected to deliver microwave EM energy from the source to the probe along a path between its first and second ports, a forward coupler 414 at the first port of the circulator 416, and a reflected coupler 418 at the third port of the circulator 416. After passing through the reflected coupler, the microwave EM energy from the third port is absorbed in a power dump load 422. The microwave signal coupling module also includes a switch 415 operated by the controller 406 via control signal V₁₂ for connecting either the forward coupled signal or the reflected coupled signal to a heterodyne receiver for detection

The other branch from the power splitter 424 forms a measurement channel. The measurement channel bypasses the amplifying line-up on the microwave channel, and hence is arranged to deliver a low power signal from the probe. In this embodiment, a primary channel selection switch 426 controlled by the controller 406 via control signal V₁₃ is operable to select a signal from either the microwave channel or the measurement channel to deliver to the probe. A high band pass filter 427 is connected between the primary channel selection switch 426 and the probe 420 to protect the microwave signal generator from low frequency RF signals.

The measurement channel includes components arranged to detect the phase and magnitude of power reflected from the probe, which may yield information about the material e.g. biological tissue present at the distal end of the probe. The measurement channel comprises a circulator 428 connected to deliver microwave EM energy from the source 402 to the probe along a path between its first and second ports. A reflected signal returned from the probe is directed into the third port of the circulator 428. The circulator 428 is used to provide isolation between the forward signal and the reflected signal to facilitate accurate measurement. However, as the circulator does not provide complete isolation between its first and third ports, i.e. some of the forward signal may break through to the third port and interfere with the reflected signal, a carrier cancellation circuit is used that injects a portion of the forward signal (from forward coupler 430) back into the signal coming out of the third port (via injection coupler 432). The carrier cancellation circuit include a phase adjustor 434 to ensure that the injected portion is 180° out of phase with any signal that breaks through into the third port from the first port in order to cancel it out. The carrier cancellation circuit also include a signal attenuator 436 to ensure that the magnitude of the injected portion is the same as any breakthrough signal.

To compensate for any drift in the forward signal, a forward coupler 438 is provided on the measurement channel. The coupled output of the forward coupler 438 and the reflected signal from the third port of the circulator 428 are connected to respective input terminal of a switch 440, which is operated by the controller 406 via control signal V₁₄ to connect either the coupled forward signal or the reflected signal to a heterodyne receiver for detection.

The output of the switch 440 (i.e. the output from the measurement channel) and the output of the switch 415 (i.e. the output from the microwave channel) are connect to a respective input terminal of a secondary channel selection switch 442, which is operable by the controller 406 via control signal V₁₅ in conjunction with the primary channel selection switch to ensure that the output of the measurement channel is connected to the heterodyne receiver when the measurement channel is supplying energy to the probe and that the output of the microwave channel is connected to the heterodyne receiver when the microwave channel is supplying energy to the probe.

The heterodyne receiver is used to extract the phase and magnitude information from the signal output by the secondary channel selection switch 442. A single heterodyne receiver is shown in this system, but a double heterodyne receiver (containing two local oscillators and mixers) to mix the source frequency down twice before the signal enters the controller may be used if necessary. The heterodyne receiver comprises a local oscillator 444 and a mixer 448 for mixing down the signal output by the secondary channel selection switch 442. The frequency of the local oscillator signal is selected so that the output from the mixer 448 is at an intermediate frequency suitable to be received in the controller 406. Band pass filters 446, 450 are provided to protect the local oscillator 444 and the controller 406 from the high frequency microwave signals.

The controller 406 receives the output of the heterodyne receiver and determines (e.g. extracts) from it information indicative of phase and magnitude of the forward and/or reflected signals on the microwave or measurement channel. This information can be used to control the delivery of high power microwave EM radiation on the microwave channel or high power RF EM radiation on the RF channel. A user may interact with the controller 406 via a user interface 452, as discussed above.

The RF channel shown in FIG. 1 comprises an RF frequency source 454 connected to a gate driver 456 that is controlled by the controller 406 via control signal V₁₆. The gate driver 456 supplies an operation signal for an RF amplifier 458, which is a half-bridge arrangement. The drain voltage of the half-bridge arrangement is controllable via a variable DC supply 460. An output transformer 462 transfers the generated RF signal on to a line for delivery to the probe 420. A low pass, band pass, band stop or notch filter 464 is connected on that line to protect the RF signal generator from high frequency microwave signals.

A current transformer 466 is connected on the RF channel to measure the current delivered to the tissue load. A potential divider 468 (which may be tapped off the output transformer) is used to measure the voltage. The output signals from the potential divider 468 and current transformer 466 (i.e. voltage outputs indicative of voltage and current) are connected directly to the controller 406 after conditioning by respective buffer amplifiers 470, 472 and voltage clamping Zener diodes 474, 476, 478, 480 (shown as signals B and C in FIG. 1).

To derive phase information, the voltage and current signals (B and C) are also connected to a phase comparator 482 (e.g. an EXOR gate) whose output voltage is integrated by RC circuit 484 to produce a voltage output (shown as A in FIG. 1) that is proportional to the phase difference between the voltage and current waveforms. This voltage output (signal A) is connected directly to the controller 406.

The microwave/measurement channel and RF channel are connected to a signal combiner 114, which conveys both types of signal separately or simultaneously along cable assembly 116 to the probe 420, from which it is delivered (e.g. radiated) into the biological tissue of a patient.

A waveguide isolator (not shown) may be provided at the junction between the microwave channel and signal combiner. The waveguide isolator may be configured to perform three functions: (i) permit the passage of very high microwave power (e.g. greater than 10 W); (ii) block the passage of RF power; and (iii) provide a high withstanding voltage (e.g. greater than 10 kV). A capacitive structure (also known as a DC break) may also be provided at (e.g. within) or adjacent the waveguide isolator. The purpose of the capacitive structure is to reduce capacitive coupling across the isolation barrier.

The present invention provides improvements to an electrosurgical apparatus.

### SUMMARY OF THE INVENTION

At its most general, the invention provides an electrosurgical apparatus having a rechargeable power source which may be charged wirelessly.

According to a first aspect of the invention, there is provided an electrosurgical apparatus comprising an oscillator for generating electromagnetic (EM) energy (e.g.

radiofrequency energy or microwave frequency energy); a controller operable to select an energy delivery profile for the oscillator; a feed structure for conveying the electromagnetic energy to an output; a rechargeable power source arranged to supply power to the oscillator; and a receiver circuit comprising an inductive coupler configured to wirelessly receive power from a transmitter and supply received power to the rechargeable power source. The selection of an energy delivery profile may involve switching the oscillator on or off in one example, or may comprise more complex operation such as the selection of a pulse profile in some embodiments.

In this way, the electrosurgical apparatus of the present invention may be charged wirelessly. This may facilitate an electrosurgical apparatus having improved ergonomics, for example by being handheld and easier to manipulate, which may be particularly important in surgical settings or environments. It is envisaged that the electrosurgical apparatus according to the present invention may not be limited to use in electrosurgery (for example cutting, coagulation, ablation and the like), but may also be used with other instruments requiring EM energy, such as sterilisation equipment (for example involving the production of a thermal or non-thermal plasma) or the like.

Advantageously, the receiver circuit may form a resonant circuit, such as, a resonant inductive circuit. For example, the receiver circuit may further comprise a capacitor and, optionally, a resistor which may be connected in series or in parallel with the inductive coupler. In this way, the receiver circuit may be configured to receive power by resonant inductive coupling, which may increase the efficiency of energy transfer from a transmitter to the receiver circuit.

Optionally, the receiver circuit may further comprise a rectifier and a regulator to convert the received alternating current (AC) signal to a direct current (DC) signal. For example, a rectifier may be a full wave bridge rectifier, a half-wave rectifier or a centre tap rectifier.

Preferably, the feed structure may comprise a transformer. For example, the transformer may transfer the generated EM energy to a line for delivery to the output. A transformer may be particularly preferable in embodiments wherein the EM energy is radiofrequency (RF) EM energy, as discussed below. Preferably, for every one turn of a primary coil of the transformer there are at least ten turns of a secondary coil of the transformer. For example, a primary coil of the transformer may have 4 turns and a secondary coil of the transformer may have 40 turns, such that there are 10 turns of the secondary coil for every turn of the primary coil. Alternatively, the primary coil of the transformer may have 15 turns and the secondary coil of the transformer may have 200 turns, such that there are more than 13 turns of the secondary coil for every turn of the primary coil. In some examples the length of each coil may be 20 mm and the diameter of each coil may be 25 mm. A capacitor may be connected to the secondary coil, for example having a capacitance of around 158 nF. For example, a resonant frequency of the secondary coil may be 400 kHz. Also, the primary coil and/or the secondary coil may be a solenoid coil (e.g. a straight core coil), for example, having an air core or a solid core. By providing a resonant frequency at 400 kHz, the transformer may be particularly suited for a frequency of operation of the electrosurgical apparatus, e.g. for performing electrosurgery, to ensure optimal power delivery from the oscillator to the output. Of course, these parameters may be varied in any other suitable way to achieve a desired resonant frequency, which may be a frequency other than 400 kHz, for example to facilitate electrosurgery or optimise wireless charging, and it is also envisaged that a tuned resonant frequency of 400 kHz may be achieved by using other values for the described parameter, or in another suitable way. In some embodiments, the transformer may have a solid core of magnetic material, e.g. ferrite or iron dust. This may be in the form of a toroidal core, for example, wherein the core may be formed of two U-shaped sections, a first section on which the primary coil is wound and a second section on which the secondary coil is wound, wherein field coupling takes place at the end of each arm of the U-shape. A solid core may be advantageous over an air core in reducing coil size or resistive losses.

Alternative numbers of turns and turns ratios may be employed in order to match the characteristics of the rechargeable power source to the required voltage and power to be delivered to a diversity of loads and load impedances at the output. In some embodiments, chokes and capacitors may be used on the primary coil and/or the secondary coil of the transformer, and may form a resonant filter structure to improve electromagnetic interference (EMI) filtering and switching characteristics. Preferably, the overall passband of such a transformer and filter structure is tuned to have a resonant peak at 400 kHz, though any suitable resonant frequency may be chosen.

Advantageously, the inductive coupler may comprise a secondary coil of the transformer. Such an arrangement allows the power source to be recharged wirelessly without requiring an additional receiver coil for wireless charging, which reduces the weight of the apparatus, further improving ergonomics of the device. Alternatively, the characteristics and parameters (e.g. length, number of turns, core-type) of the secondary coil of a transformer as discussed herein may be used for the inductive coupler of the receiver circuit.

Optionally, the apparatus may comprise a radiofrequency (RF) electromagnetic energy generator, and the feed structure may comprise a radiofrequency channel to convey the microwave frequency EM energy to the output. For example, the radiofrequency channel may be adapted for conveying RF EM energy, and may comprise any or all features of an RF channel as described above with respect to FIG. 1. In this way the electrosurgical apparatus may be adapted for delivering RF energy to an electrosurgical instrument. In some embodiments, certain components of the RF channel of FIG. 1 may be omitted. For example, the controller 406 may provide some of the functionality provided by some other components (e.g. components 470, 472, 474, 476, 478, 480, 482, 484) such that these other components can be omitted without reducing functionality.

Additionally or alternatively, the apparatus may comprise a microwave frequency EM energy generator, and the feed structure may comprise a microwave channel to convey the microwave frequency EM energy to the output. For example, the microwave frequency channel may be adapted for conveying microwave EM energy, and may comprise any features of a microwave channel as described above with respect to FIG. 1. As mentioned above in respect of the RF channel, in some embodiments, certain hardware components of the microwave channel may be omitted and their functionality may be performed by controller software instead.

In embodiments where each of a RF EM energy generator and microwave frequency EM energy generator are present, the RF channel and the microwave channel may comprise physically separate signal pathways for conveying the respective RF and microwave energy. In some examples, the feed structure may comprise a signal combiner (which may also be referred to herein as a power combiner) for conveying both the RF and the microwave frequency EM energy to the output.

For example, the oscillator may be a RF oscillator or a microwave frequency oscillator, and may form part of the RF EM energy generator or the microwave frequency EM energy generator, respectively. That is, the electrosurgical apparatus may comprise only an RF EM energy generator, and so the oscillator may form part of the RF EM energy generator. Alternatively, the electrosurgical apparatus may comprise only a microwave EM energy generator, and so the oscillator may form part of the microwave EM energy generator. In other embodiments, each of an RF EM energy generator and a microwave EM energy generator may be present, such that the oscillator may form part of either the RF EM energy generator or the microwave EM energy generator, as required. For example, the oscillator may be capable of generating only one of RF EM energy and microwave frequency EM energy, and a second oscillator may be provided that is capable of generating the other one of RF EM energy and microwave frequency EM energy. The second oscillator may receive power from the rechargeable power source, and may be operated by the controller, and may be analogous to the oscillator. Alternatively, the oscillator may be capable of generating both RF EM energy and microwave frequency EM energy, and no second oscillator may be present.

Preferably, the rechargeable power source is a battery, though a capacitor or a supercapacitor may also be used. For example, the battery may be a lithium-ion battery, or a lithium-ion polymer or lithium polymer (LiPo) battery. The choice of power source may depend on the desired characteristics of the device. For example, a power source may be chosen based on its ability to provide a higher current or a higher voltage. In some examples, the apparatus may comprise a DC-DC converter which may change the supply voltage from the power source, for example to vary the output power or make better use of power as the power source voltage drops upon discharge.

Preferably, the electrosurgical apparatus further comprises a switching circuit to switch the rechargeable power source between a first mode for receiving power from the receiver circuit a second mode and providing power to the oscillator. For example, the controller may be configured to operate the switching circuit, or the switching circuit may be operated independently of the controller.

Preferably, the receiver circuit may also be configured to allow wired charging of the rechargeable power source, in addition to wireless charging using the inductive coupler. For example, to allow wired charging the receiver circuit may comprise a connector to receive energy for charging the rechargeable power source. In one embodiment, the connector may be provided in the form of one or more galvanic contacts, or any other suitable electrical connector. Additionally or alternatively, the output may be configured to provide the connector, such that the rechargeable power source may be charged by delivering energy into the electrosurgical apparatus to the receiver circuit via the output. By configuring the receiver circuit in this way, the rechargeable power source may additionally be recharged without using wireless charging, wherein wired charging can provide faster charging speeds which may be desirable in certain circumstances. For example, clinical conditions (e.g. sterility) may dictate that the rechargeable power source should be charged either wirelessly or by a wired connection. Wired charging may use mains power, for example. Optionally, the connector may be adapted to receive a fast-charge current to charge the rechargeable power source via the receiver circuit.

Optionally, the electrosurgical apparatus may comprise an electrosurgical instrument connected to receive electromagnetic energy from the output and, possibly, configured to deliver the received electromagnetic energy into biological tissue, for example, at a treatment site on or in a patient. For example the electrosurgical instrument may be detachably connected to the output, via a QMA connector or the like, to allow the electrosurgical apparatus to be used with a variety of electrosurgical instruments. Alternatively, the electrosurgical instrument may be unitary with the electrosurgical apparatus. In certain embodiments the electrosurgical instrument may be a cutting instrument, a coagulating instrument, an ablation instrument or any other instrument which may use EM energy, such as RF or microwave EM energy. Preferably, the electrosurgical instrument may comprise a bipolar coaxial cutting tool and, for example, the electrosurgical apparatus with the instrument may be capable of producing a 400 kHz 150 W continuous wave signal that can be used for cutting tissue. Other electrosurgical instruments may also be considered, for example an electrosurgical instrument which may be configured to generate a thermal or non-thermal plasma. In some examples, the electrosurgical instrument may comprise a coaxial cable and a probe tip mounted at a distal end of the coaxial cable, wherein the probe tip may radiate EM energy to tissue.

Advantageously, the electrosurgical apparatus may comprise a housing which is adapted to be handheld by a user. The housing may include enclose (e.g. completely) the oscillator, the controller, the feed structure, the rechargeable power source, and the receiver circuit. Where the electrosurgical apparatus includes an electrosurgical instrument, the housing may not enclose some or all of the instrument.

According to a second aspect of the invention, there is provided an electrosurgical system comprising an electrosurgical apparatus as described above with respect to the first aspect of the invention, and a transmitter for wirelessly providing power to the electrosurgical apparatus.

Preferably, the transmitter may comprise a transmitter circuit having an inductive coupler arranged to transmit power to the receiver circuit via inductive coupling (e.g. non-resonant inductive coupling). In some examples, the power may be delivered wirelessly to the electrosurgical apparatus by resonant inductive coupling, wherein the receiver circuit, and in some examples also the transmitter circuit, is a resonant circuit.

Optionally, the transmitter may comprise a housing which is adapted to receive a portion of the electrosurgical apparatus. For example, the housing of the apparatus and the housing of the transmitter may have corresponding interlocking parts to hold them in a fixed relative position which ensures maximum efficiency of power transfer between the transmitter and the electrosurgical apparatus.

Optionally, the electrosurgical system may further include a wired charger configured to form a wired electrical connection with the electrosurgical apparatus. The wired charger may be configured to deliver power non-wirelessly to the electrosurgical apparatus, for example, to recharge a power source of the electrosurgical apparatus. The wired connection may include one or more galvanic contacts, or any other suitable electrical connector. Additionally or alternatively, the output may be configured to provide the connector, such that the rechargeable power source may be charged by delivering energy into the electrosurgical apparatus to the receiver circuit via the output.

As used herein, the term “receiver circuit” is generally used to denote any circuitry which is involved in charging of the rechargeable power source. This may include features which are provided only for charging of the rechargeable power source (such as an inductive coupler in some embodiments), as well as features which also perform other functions (such as the output where it may also form a connector for wired charging, a secondary coil of a transformer where it is used as an inductive coupler for wireless charging).

Herein, the term “inner” means radially closer to the centre (e.g. axis) of the coaxial cable, probe tip, and/or applicator. The term “outer” means radially further from the centre (axis) of the coaxial cable, probe tip, and/or applicator.

The term “conductive” is used here to mean electrically conductive, unless the context dictates otherwise.

Herein, the terms “proximal” and “distal” refers to the ends of the applicator. In use, the proximal end is closer to a generator for providing the RF and/or microwave energy, whereas the distal end is further from the generator.

In this specification “microwave” may be used broadly to indicate a frequency range of 400 MHz to 100 GHz, but preferably in the range 1 GHz to 60 GHz. Specific frequencies that have been considered are: 915 MHz, 2.45 GHz, 3.3 GHz, 5.8 GHz, 10 GHz, 14.5 GHz, and 25 GHz. In contrast, this specification uses “radiofrequency” or “RF” to indicate a frequency range that is at least three orders of magnitude lower, e.g. up to 300 MHz, preferably 10 kHz to 1 MHz, and most preferably 400 kHz. The microwave frequency may be adjusted to enable the microwave energy delivered to be optimised. For example, a probe tip may be designed to operate at a certain frequency (e.g. 900 MHz), but in use the most efficient frequency may be different (e.g. 866 MHz).

The term “electrosurgical” is used in relation to an instrument, apparatus, or tool which is used during surgery and which utilises radiofrequency and/or microwave frequency electromagnetic (EM) energy.

### BRIEF DESCRIPTION OF THE DRAWINGS

Features of the invention are now explained in the detailed description of examples of the invention given below with reference to the accompanying drawings, in which:

FIG. 1 is an overall schematic diagram of a prior art electrosurgical apparatus, and is discussed above;

FIG. 2 is a simplified schematic diagram of an electrosurgical apparatus;

FIG. 3 is a schematic diagram of an electrosurgical system according to a first embodiment of the present invention;

FIG. 4 is a schematic diagram of an electrosurgical system according to a second embodiment of the present invention;

FIG. 5 is a schematic diagram of an electrosurgical system according to a third embodiment of the present invention;

FIG. 6 is a schematic diagram of an electrosurgical system according to a fourth embodiment of the present invention;

FIG. 7 is a schematic diagram of a transmitter which may be used in embodiments of the present invention;

FIGS. 8a and 8b shows an electrosurgical system according to an embodiment of the present invention;

FIGS. 9a and 9b show cut through images of an electrosurgical apparatus according to embodiments of the present invention; and

FIG. 10 is a schematic circuit diagram of an electrosurgical system according to another embodiment of the present invention.

### DETAILED DESCRIPTION; FURTHER OPTIONS AND PREFERENCES

This invention relates to electrosurgical apparatus having a rechargeable power source which may be charged wirelessly.

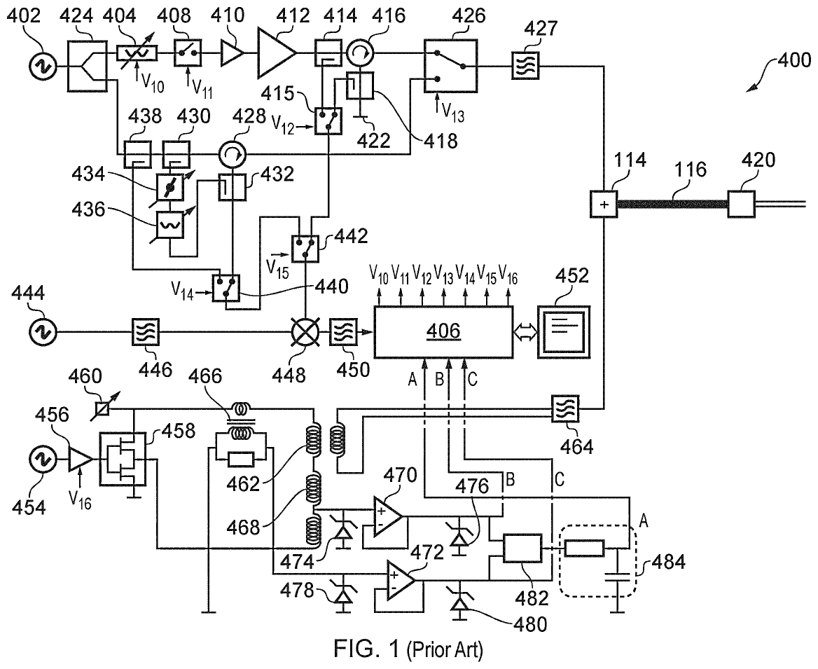

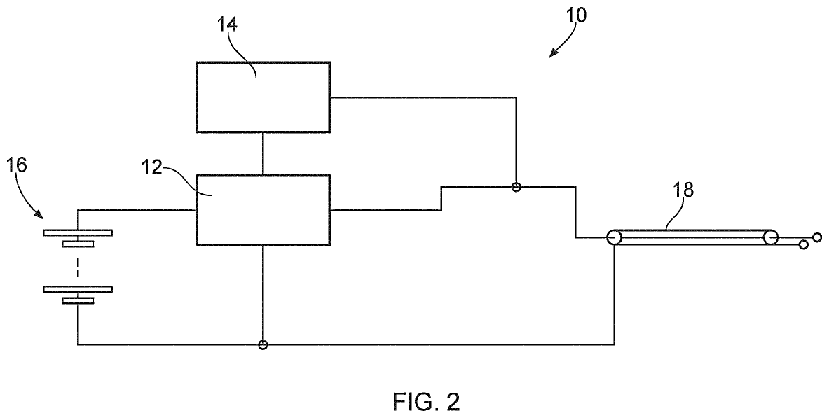

FIG. 2 shows a simplified schematic diagram of an electrosurgical apparatus 10, with respect to which the advantages of the present invention will be described below. In general, the schematic shows a simplified version of an electrosurgical apparatus 10 which is similar to that described above with respect to FIG. 1. However, the electrosurgical apparatus 10 comprises only a single oscillator 12 for producing radiofrequency (RF) or microwave frequency electromagnetic (EM) energy, and so the apparatus 10 comprises only one of an RF channel or a microwave channel, whereas the apparatus 400 comprises both a RF channel and a microwave channel.

Other components such as amplifiers, power splitters and the like, for example as discussed above with respect to FIG. 1, may be present to manipulate the RF or microwave EM energy, and/or to monitor the RF or microwave energy which is delivered and/or reflected, but are omitted in FIG. 2 for clarity. In particular, in examples where the oscillator 12 is configured to produce RF EM energy, the apparatus 10 may comprise a transformer in the RF channel to transfer the RF signal on to a line for delivery to a coaxial cable 18. For example, the coaxial cable 18 may form part of an electrosurgical instrument, or may be provided to deliver energy to an electrosurgical instrument. In certain embodiments, the coaxial cable 18 may be detachably connected to the apparatus 10, for example by a QMA connector or the like.

A controller 14 is provided, which may be configured to perform many of the functions as discussed above with respect to FIG. 1, but in particular the controller 14 is operable to select an energy delivery profile for the oscillator 12. The controller 14 may also monitor radiation which is transmitted and/or reflected from an electrosurgical instrument. For example, in embodiments where RF EM energy is supplied, the controller 14 may monitor current and voltage of a transmitted signal. In embodiments where microwave EM energy is supplied, the controller 14 may monitor transmitted and reflected signals.

The electrosurgical apparatus 10 comprises a rechargeable power source 16 for supplying energy to the oscillator 12. For example, the rechargeable power source 16 may comprise a battery, such as a lithium-polymer battery, though any suitable rechargeable power supply may be considered, such as a capacitor or supercapacitor. As the electrosurgical apparatus 10 comprises an internal power source 16 which is rechargeable, the apparatus 10 is easily portable and more convenient when compared with apparatuses or generators which require mains power to operate. The present invention is particularly concerned with means for wirelessly charging the power source 16.

The oscillator 12 is connected to a coaxial cable 18 via a feed structure, wherein the feed structure may form part of the RF or microwave channel. The coaxial cable 18 is used to convey electrosurgical energy to an electrosurgical instrument (not shown). For example, the electrosurgical apparatus 10 may be used with a probe which is able to perform cutting, dissection, coagulation or ablation of biological tissue using the RF or microwave energy, and may be used to generate plasma for treating tissue or for sterilisation more generally (e.g. sterilisation of devices and machines).

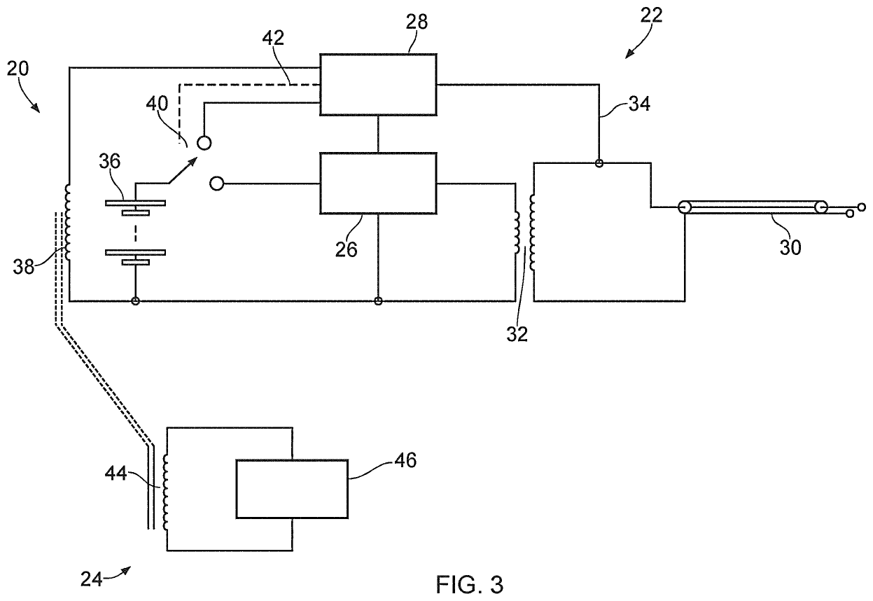

FIG. 3 shows a schematic diagram of an electrosurgical system 20 which is an embodiment of the present invention. The electrosurgical system 20 comprises an electrosurgical apparatus 22 and a transmitter 24 for wirelessly providing power to the electrosurgical apparatus 22.

The electrosurgical apparatus 22 comprises an oscillator 26 for producing radiofrequency (RF) energy. A controller 28 is operable to select an energy delivery profile for the oscillator 26, as well as controlling other functions of the apparatus 22. For example, the controller 28 may be operable to turn the oscillator 26 off and on. A feed structure conveys the RF energy to a coaxial cable 30, which may be used to deliver the RF energy to an electrosurgical instrument. The feed structure comprises a transformer 32 to transfer the generated RF signal to the coaxial cable 30. In some embodiments, the feed structure may comprise a twisted pair cable to convey energy from a secondary coil of the transformer 32 to the coaxial cable 30. A feedback path 34 from the coaxial cable 30 is connected to the controller 28 to enable the controller 28 to monitor current and voltage of the RF signal which is conveyed to the output and adjust the output of the oscillator 26 accordingly. Other features of an

RF channel, for example as discussed above with respect to FIG. 1, may also be present, but are omitted in FIG. 3 for clarity. A rechargeable power source 36 provides power for the oscillator 26. To recharge the power source 36, the apparatus 22 comprises a receiver circuit 38 for wirelessly receiving power from the transmitter 24. The receiver circuit 38 comprises an inductive coupler, for example comprising an inductor in the form of a coil of wire, for receiving power by inductive coupling from a corresponding inductive coupler in the transmitter 24. For example, the coil of wire may comprise 200 turns, and may have a length of 25 mm and a diameter of 20 mm. In certain embodiments, the coil of wire may be wrapped around a core, which is preferably made of a magnetic material such as ferrite or an iron powder core. Of course, the parameters of the inductive coupler may be varied such that the inductive coupler may take any suitable form. In some examples the core may be provided generally in a U-shape, which may correspond with a matching U-shaped core of a coil in the transmitter 24 (such that the transmitter and receiver cores form a generally toroidal shape when positioned together for wireless power transfer), to increase efficiency of energy transfer between the transmitter 24 and the receiver circuit 38. Of course, it is envisaged that the core may be provided in any suitable shape. The controller 28 is configured to operate a switch 40 via a control line 42. By operating the switch 40, the power source 36 can be selectively connected with the oscillator 26 in an operating mode, for example to perform electrosurgery, or the receiver circuit 38 in a recharging mode, for example to charge the rechargeable power source 36.

In some examples, the receiver circuit 38 may additionally comprise a capacitor and, optionally, a resistor which may be connected in series or in parallel with the inductive coupler such that the receiver circuit forms a resonant inductive circuit. For example, for resonance at 400 kHz, a capacitance of 158 nF may be used (C=1/((2π×400×10³)²×1×10⁻⁶), though any combination of capacitor and resistor may be chosen to obtain desirable resonant characteristics. For example, the receiver circuit 38 may be configured to resonate at any suitable frequency, and 400 kHz is given only by way of example. By providing a circuit and, optionally, a resistor in this way, the receiver circuit 38 may be configured to receive power from the transmitter 24 by resonant inductive coupling. Advantageously, the receiver circuit 38 may also comprise a rectifier and a regulator to convert a received voltage from AC to DC.

The inductive coupler is preferably positioned near a sidewall of a housing of the electrosurgical apparatus 22. In this way, the coil is positioned in a manner which ensures that, when the electrosurgical apparatus 22 is suitably positioned relative to the transmitter 24, substantially all of the magnetic field generated by the transmitter 24 passes through the secondary coil, maximising efficiency of power transfer between the transmitter 24 and the electrosurgical apparatus 52.

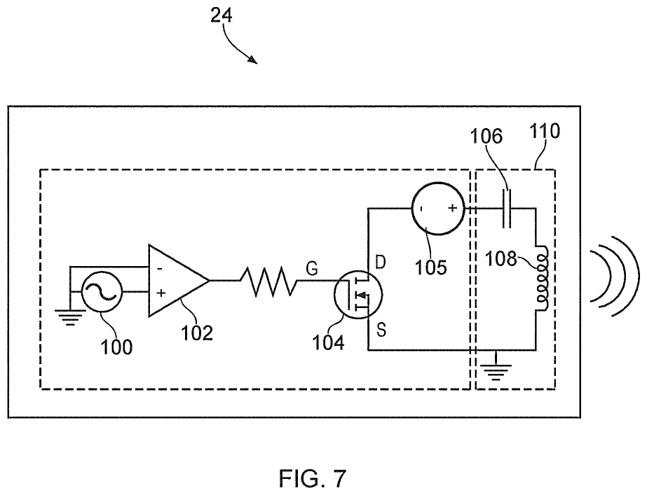

The transmitter 24 also comprises an inductive coupler 44 which is configured to receive power from a charging source 46 to generate an oscillating magnetic field and thereby induce a current in the corresponding inductive coupler of the receiver circuit 38. The charging source 46 may comprise mains power or a battery pack, for example. An example of a transmitter which may be used in the electrosurgical system 20 is shown in FIG. 7.

In addition to monitoring current and voltage of the RF signal, the controller 28 may also be configured to monitor charging and discharging of the rechargeable power source 36. For example, the controller 28 may comprise a charge balancing circuit, an over temperature cut out and other features to form a battery management system to help maximise the life of the rechargeable power source 36. In an embodiment, the controller 28 may include a rectification circuit to convert a received voltage from AC to DC. It is to be understood that in some embodiments the coil of the receiver circuit 38 may have a different type of core to the coil of the transmitter 24. For example, one coil may have an air core and the other coil may have a solid core (e.g. iron powder/dust core). Alternatively, both cores may be the same, e.g. an air core or a solid core.

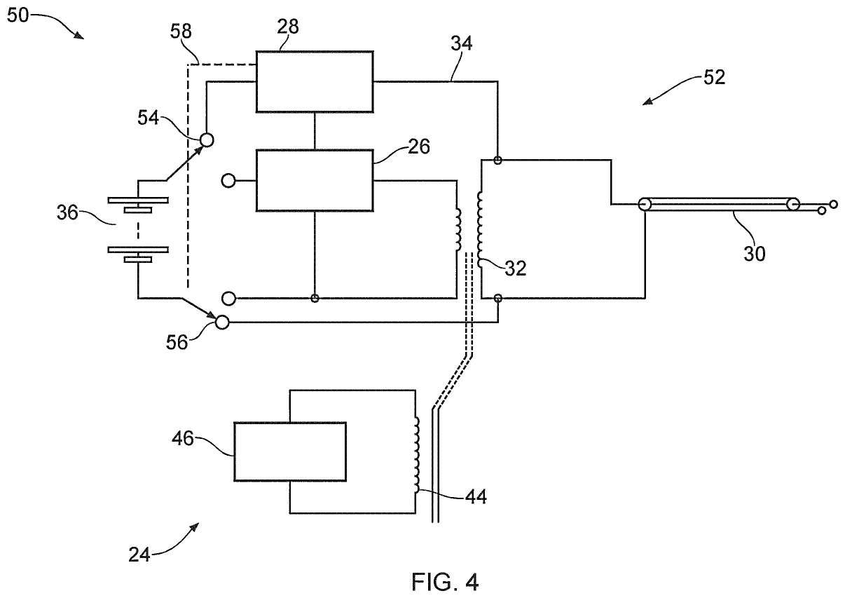

FIG. 4 shows a schematic diagram of a second electrosurgical system 50 which is a further embodiment of the present invention. Components which are equivalent to those described above are given corresponding reference numerals, and description thereof is not repeated.

The electrosurgical system 50 comprises an electrosurgical apparatus 52 and a transmitter 24. The transmitter 24 may be a transmitter 24 as shown in FIG. 7, for example.

In this embodiment, the electrosurgical apparatus 52 does not include a dedicated inductive coupler for wirelessly receiving power from the transmitter 24. Instead, a secondary coil of the transformer 32 is used to perform this function. The inductive coupler 44 of the transmitter 24 receives power from the charging source 26 to generate an oscillating magnetic field, and thereby induce a current in the second coil of the transformer 32. In some examples, a capacitor and, optionally, a resistor may be connected to the secondary coil of the transformer 32, either in series or in parallel, to form a resonant inductive circuit, as described above with respect to FIG. 3. The controller 28 is configured to operate switches 54, 56 via a control line 58 to selectively connect the rechargeable power source 36 to the secondary coil of the transformer 32 for charging by the induced current. In an operating mode, the controller 28 can operate the switches 54, 56 to electrically connect the power source 36 to the oscillator 26 to generate RF EM energy for electrosurgery.

Although not shown, additional circuitry such as chokes and capacitors may be connected to the primary and/or secondary coils of the transformer 32 to filter out electromagnetic interference (EMI) and improve switching characteristics. In certain embodiments, each of the primary and secondary coils of the transformer 32 may be an air-cored solenoid having a diameter of 25 mm and a length of 20 mm. The primary coil may have 15 turns, and the secondary coil may have 200 turns. A capacitor of around 158 nF may be connected to the secondary coil. In this way, the transformer 32 may have a tuned resonant frequency of 400 kHz, which is particularly suitable for use as a receiver for wireless charging, for example in combination with the transmitter 24. Of course, these parameters may be varied in any other suitable way to achieve a desired resonant frequency, which may be a frequency other than 400 kHz, and it is also envisaged that a tuned resonant frequency of 400 kHz may be achieved by using other values for the described parameters, or in another suitable way.

By using the secondary coil as a receiver for wireless charging, the larger number of turns compared with the primary coil means that a higher voltage can be obtained from a flux linked from the transmitter 24. Of course, the transformer 32 may comprise other core materials, preferably a magnetic material such as ferrite or an iron powder or dust.

By using the secondary coil of the transformer 32 for wireless charging of the power source 36 in this way, no dedicated wireless charging coil is required. This keeps the weight and size of the components of the electrosurgical apparatus 52 small, enabling portability and, in some examples, the electrosurgical apparatus 52 may be hand-held.

To allow the secondary coil of the transformer 32 to be used as an inductive coupler for wireless charging, the transformer 32 is preferably positioned near a sidewall of a housing of the electrosurgical apparatus 52. In this way, the secondary coil is positioned in a manner which ensures that, when the electrosurgical apparatus 52 is suitably positioned relative to the transmitter 24, substantially all of the magnetic field generated by the transmitter 24 passes through the secondary coil, maximising efficiency of power transfer between the transmitter 24 and the electrosurgical apparatus 52. The primary coil of the transformer 32 will receive a much lower induced voltage when charging than the secondary coil. However, in some examples, the controller 28 may comprise circuitry to protect components connected to the primary coil side of the transformer 32 when the apparatus is charging.

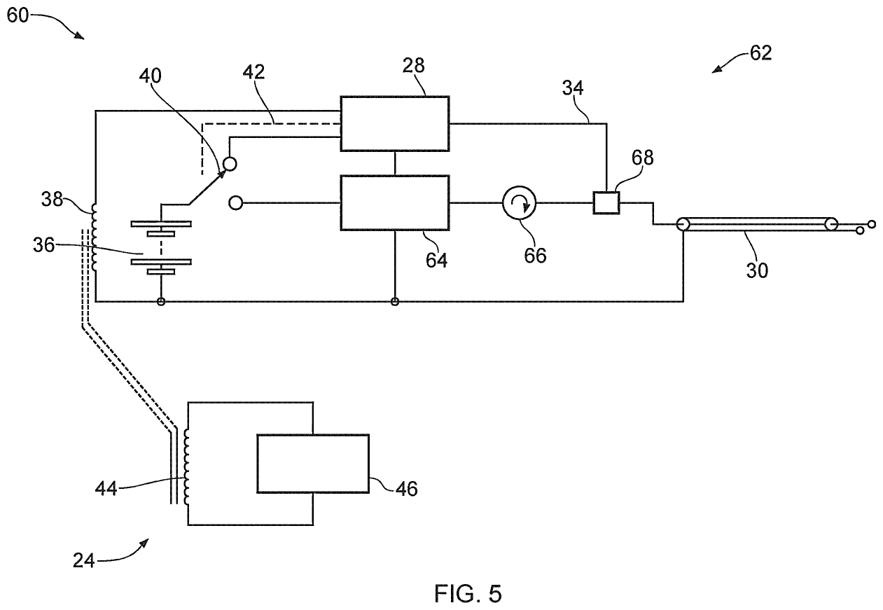

FIG. 5 shows a schematic diagram of a third electrosurgical system 60 which is a further embodiment of the present invention. Components which are equivalent to those described above are given corresponding reference numerals, and description thereof is not repeated.

The electrosurgical system 60 comprises an electrosurgical apparatus 62 and a transmitter 24. In this embodiment, the electrosurgical apparatus 62 comprises an oscillator 64 which is configured to generate microwave frequency electromagnetic (EM) energy for delivery to an electrosurgical instrument via a coaxial cable 30. The electrosurgical apparatus 62 therefore comprises a microwave channel between the oscillator 64 and the coaxial cable 30, but no RF channel. Features of a microwave channel as described above with respect to FIG. 1 may therefore be included in some arrangements, but are omitted from FIG. 5 for clarity. The transmitter 24 may be a transmitter as described below with respect to FIG. 7, for example.

The microwave channel comprises a circulator 66 connected to deliver microwave EM energy from the oscillator 64 to the coaxial cable 30 along a path between its first and second ports. A third port (not shown) of the circulator 66 may be connected to a reflected coupler to be absorbed in a power dump load, for example, as described above with respect to FIG. 1. A coupler 68 is provided in the microwave channel, which directs a portion of a reflected signal to the controller 28 to allow the controller 28 to monitor and analyse reflected signals via the feedback path 34. For example, the operation of coupler 68 may be analogous to that of coupler 414 and/or 418 of FIG. 1. Of course, it is envisaged that other methods of feedback or measurement of the microwave channel may be considered as an alternative, or in addition to, those methods described herein. For example, in some embodiments, coupler 68 may be omitted.

The electrosurgical apparatus 62 comprises a receiver circuit 38 configured to recharge the rechargeable battery 36 using energy received from a transmitter 24 in substantially the same manner as described above with respect to FIG. 3.

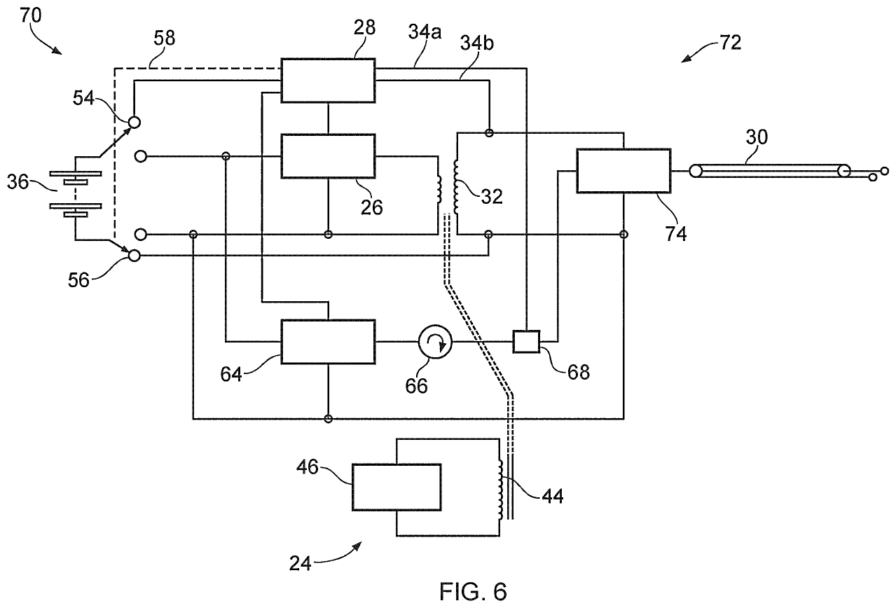

FIG. 6 is a schematic diagram of an electrosurgical system 70 according to a fourth embodiment of the present invention. Components which are equivalent to those described above are given corresponding reference numerals, and description thereof is not repeated.

The electrosurgical system comprises an electrosurgical apparatus 72 and a transmitter 24. In this embodiment, the electrosurgical apparatus 72 comprises both an RF oscillator 26 and a microwave frequency oscillator 64, which are each configured to supply energy to a coaxial cable 30. The electrosurgical apparatus therefore comprises an RF channel configured to convey RF energy from the RF oscillator 26 to the coaxial cable 30, and a microwave channel configured to convey microwave frequency energy from the microwave oscillator 64 to the coaxial cable 30. The RF channel and the microwave channel may each comprise components as discussed above with respect to FIG. 1, in some examples, as well as components discussed above with respect to FIGS. 3-5. The electrosurgical apparatus 72 comprises a combiner 74 which is configured to take RF energy from the RF channel and microwave frequency energy from the microwave channel and combine them onto a single output to be delivered to the coaxial cable 30. The controller 28 is configured to monitor the microwave frequency energy delivered to and reflected via the coaxial cable 30 through a microwave feedback channel 34a, and monitor RF energy delivered to the coaxial cable through an RF feedback channel 34b.

In this embodiment, the electrosurgical apparatus 72 may receive power wirelessly from the transmitter 24 for charging the battery 36 using a secondary coil of a transformer 32 on the RF channel, as described above with respect to FIG. 4.

The electrosurgical system 70 thereby provides an electrosurgical apparatus 72 for delivering RF and/or microwave frequency EM energy, and which is rechargeable wirelessly. The electrosurgical apparatus 72 is therefore more convenient, and may be used in situations where a portable apparatus is advantageous.

FIG. 7 shows a schematic diagram of a transmitter 24 which may be used with embodiments of the present invention. For example, the transmitter 24 may be positioned in a charging cradle which is used to charge an electrosurgical apparatus.

As seen in FIG. 7, an oscillator 100 provides an oscillating control signal to an amplifier 102. The oscillating control signal may be an oscillating voltage signal having a frequency in the MHz range (e.g. 9.9 MHz). The amplifier 102 amplifies this oscillating control signal to form an oscillating drive signal which has the same frequency as the oscillating control signal but is more powerful such that the oscillating drive signal possesses enough power to drive a MOSFET 104. Specifically, the MOSFET 104 is a voltage controlled current source and, therefore, generates an oscillating current signal (using current supply 105) based on the oscillating drive signal. The oscillating current signal has the same frequency as the control signal and drive signal. This oscillating current signal is then provided to the primary (or transmitter) inductive coupler 110. The primary inductive coupler 110 uses the oscillating current signal to generate an oscillating magnetic field via electromagnetic induction.

The primary inductive coupler 110 comprises a series inductor-capacitor (LC) circuit having capacitor 106 and inductor 108. It is to be understood that the inductor 108 comprises a coil of wire, which in some embodiments may be wound on a core material. As such, the primary inductive coupler 110 is a resonant circuit. The specific values of the frequency of the oscillator 100, the capacitance of the capacitor 106 and inductance of the inductor 108 are chosen such that resonance occurs. Resonance may be set to occur based on parameters set by the physical geometry of the transmitter and receiver. In this way, the coil of the inductor 108 generates an oscillating magnetic field. The oscillating magnetic field may be used to induce a current in a corresponding inductive coupler within an electrosurgical apparatus as described above, and so recharge a rechargeable power source 36. It is to be understood that the inductive coupler 110 may be a non-resonant inductive coupler in some embodiments.

In certain embodiments, the primary inductive coupler 110 is located near a sidewall of a housing of the transmitter 24 to ensure that substantially all of the magnetic field generated by the transmitter 24 passes through the receiver coil of an electrosurgical apparatus (such as described above with respect to FIGS. 3-6), maximising efficiency of power transfer between the transmitter 24 and the electrosurgical apparatus. The primary inductive coupler 110 may comprise a coil of wire wound on a magnetic core material, such as ferrite or an iron powder core. In some examples, the core may be generally U-shaped so as to correspond with an inductive coupler of a receiver circuit within an electrosurgical apparatus such that the two U-shaped cores are positioned together for wireless power transfer to form a generally toroidal shape.

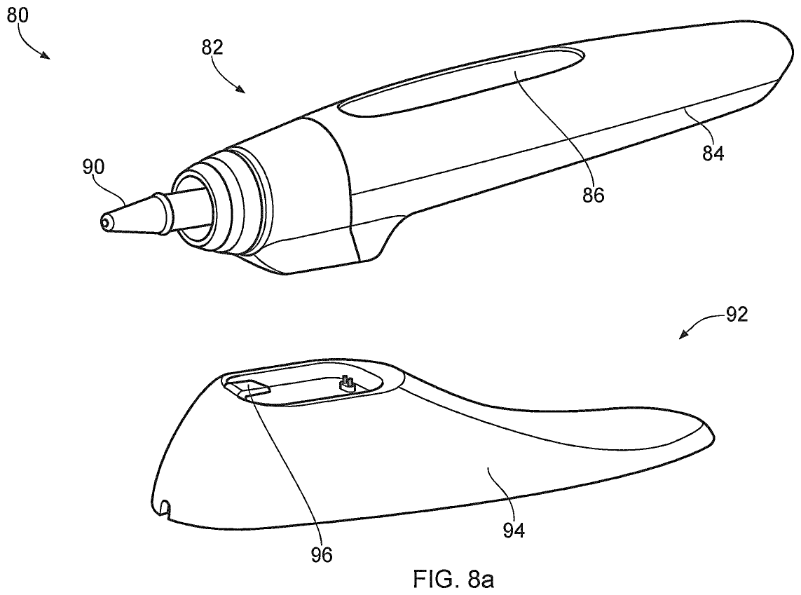

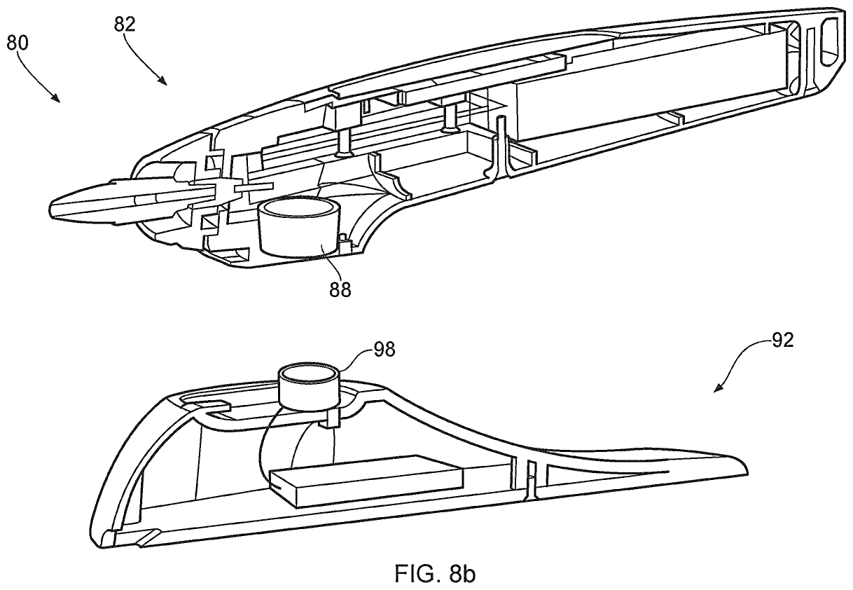

FIG. 8a shows an image of an electrosurgical system 80 according to an embodiment of the present invention. The electrosurgical system 80 comprises an electrosurgical apparatus 82 and a transmitter 92. FIG. 8b shows a cut-through image showing the placement of charging coils in the arrangement of FIG. 8a.

The electrosurgical apparatus 82 may be an electrosurgical apparatus as described above with respect to any of FIGS. 3 to 6, for example. In particular, the electrosurgical apparatus 82 comprises a housing 84 which contains a circuit for producing electrosurgical energy as shown in any of FIGS. 3 to 6. The housing 84 is preferably sized and shaped to be handheld by a user for performing electrosurgery or the like. On an upper surface of the housing 84 there is provided a control panel 86 for the apparatus 82. For example the control panel 86 may have an on/off button which is operable by a user to activate the RF and/or microwave frequency oscillator to generate EM energy for electrosurgery. The on/off button may be connected to a controller within the apparatus 82 to choose operating modes of the apparatus 82. In some embodiments the on/off button may be operable by a user to cycle through modes, such as an RF only mode, a microwave only mode, and/or a mode in which both RF and microwave frequency EM energy is generated. In some embodiments, when the electrosurgical apparatus is turned off, the controller is configured to operate switches within the apparatus 82 to connect a rechargeable battery within the apparatus 82 to a receiver circuit, as discussed with respect to FIGS. 3 to 6 above.

The outer surface of the housing, and in particular the control panel 86, may also contain other visual displays, for example a battery status indicator, which may be provided by a screen or by an LED, for example. The battery status indicator allows a user to see the amount of charge left within the rechargeable battery and so indicates when charging may be needed, or when the battery is fully charged or is charging, for example. Other visual displays or indicators, or audible, vibrational or haptic transducers may be present on the housing 84 or within the apparatus 82 as appropriate.

As shown in FIG. 8b, the electrosurgical apparatus 82 comprises a receiving inductive coupler 88 within the housing 84 for receiving energy wirelessly from the transmitter 92. In particular, the inductive coupler 88 is a coil of wire. For example, the inductive coupler 88 may be a dedicated coil for wireless charging or may form part of a transformer, as described above with respect to FIGS. 3 to 6. In some embodiments, the coil of wire may be wound on a solid core, for example of a magnetic material such as ferrite or an iron powder core. The inductive coupler 88 is positioned at a lower side of the housing 84 in order to maximise inductive coupling with a transmitting inductive coupler 98 within the transmitter 92.

The electrosurgical apparatus 82 further comprises an electrosurgical instrument 90 which may be used to perform electrosurgery. For example, the electrosurgical instrument 90 may be used to cut and/or ablate biological tissue. The instrument 90 is connected to an output of the circuit within the housing 84, for example as discussed above with respect to FIGS. 3-6, in order to receive generated EM energy. The electrosurgical instrument 90 may be detachably mounted to the housing 84, or in some embodiments may be a permanent fixture thereof.

The transmitter 92 is provided as a docking station or cradle for the electrosurgical apparatus 82, and transmits energy wirelessly to the electrosurgical apparatus 82 for charging a battery thereof. The transmitter 92 comprises a housing 94, an upper surface of which is adapted to receive the electrosurgical apparatus 82 when the apparatus 82 is not in use. The housing 94 may contain a circuit as shown in FIG. 7, for example, for recharging the apparatus 82. The housing 94 comprises a projection 96 which engages a corresponding recess in the housing 84 of the apparatus 82. The housing 94 thereby holds the apparatus 82 in an optimal position for charging, and the projection 96 helps to ensure that the apparatus 82 is not accidentally knocked from the transmitter 92, and so ensures continuity of charging.

As shown in FIG. 8b, the transmitter comprises a transmitting inductive coupler 98 positioned within the housing 94 for transmitting energy wirelessly to the electrosurgical apparatus 82. In particular, the inductive coupler 98 is a coil of wire. In some embodiments, the coil of wire may be wound on a solid core, for example of a magnetic material such as ferrite or an iron powder core. The inductive coupler 98 is positioned at an upper side of the housing 84 in order to maximise inductive coupling with the receiving inductive coupler 86 within the electrosurgical apparatus 82.

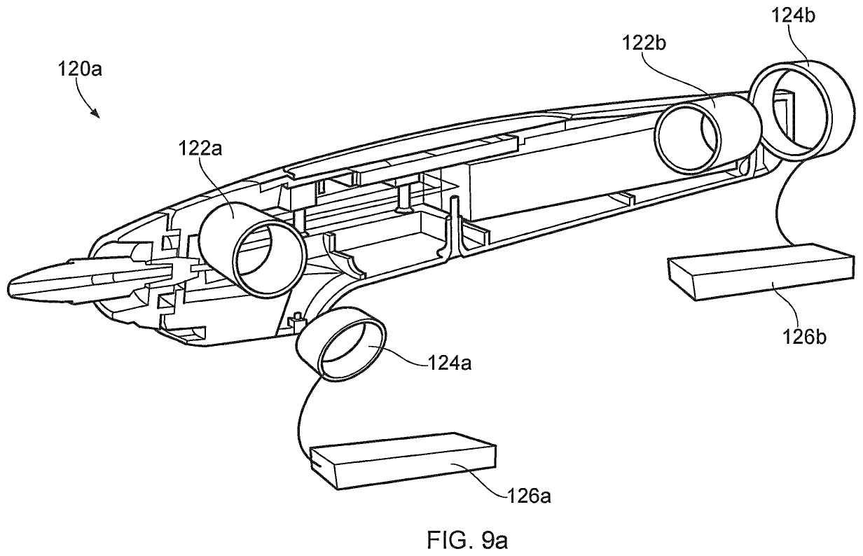

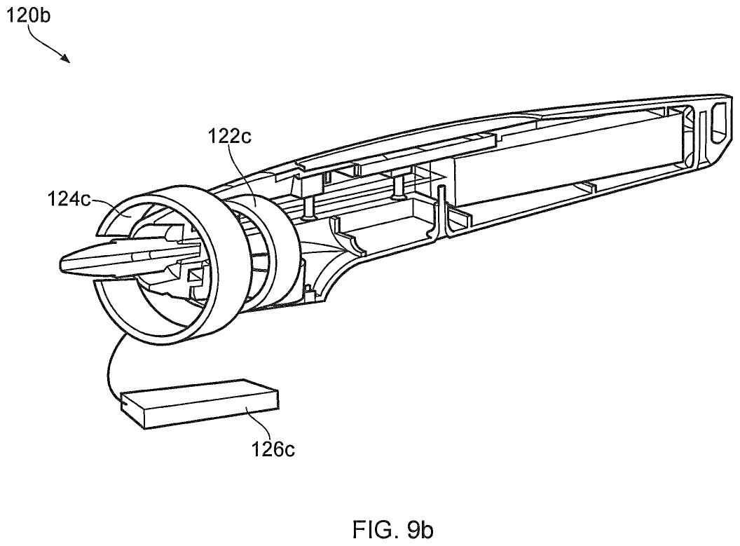

FIGS. 9a and 9b are cross-sectional views of an electrosurgical apparatus 120a, 120b showing alternative positions of a receiving inductive coupler 122a, 122b, 122c within the apparatus 120a, 120b. The electrosurgical apparatus 120a, 120b may comprise any of the features of an electrosurgical apparatus as described above. A transmitting inductive coupler 124a, 124b, 124c is also shown, connected to a transmitter circuit 126a, 126b, 126c. The transmitting inductive coupler 124a, 124b, 124c and the transmitter circuit 126a, 126b, 126c may be housed within a transmitter, such as that shown in FIGS. 8a and 8b, for example, and may comprise any of the features of a transmitter as described above. Of course, it will be understood that in preferred embodiments only one of the inductive couplers 122a, 122b, 122c may be present in an electrosurgical apparatus 120a, 120b. It will also be understood that receiving inductive coupler 122a is positioned for use with transmitting inductive coupler 124a, and transmitting circuit 126a. The remaining inductive couplers corresponding in a like manner.

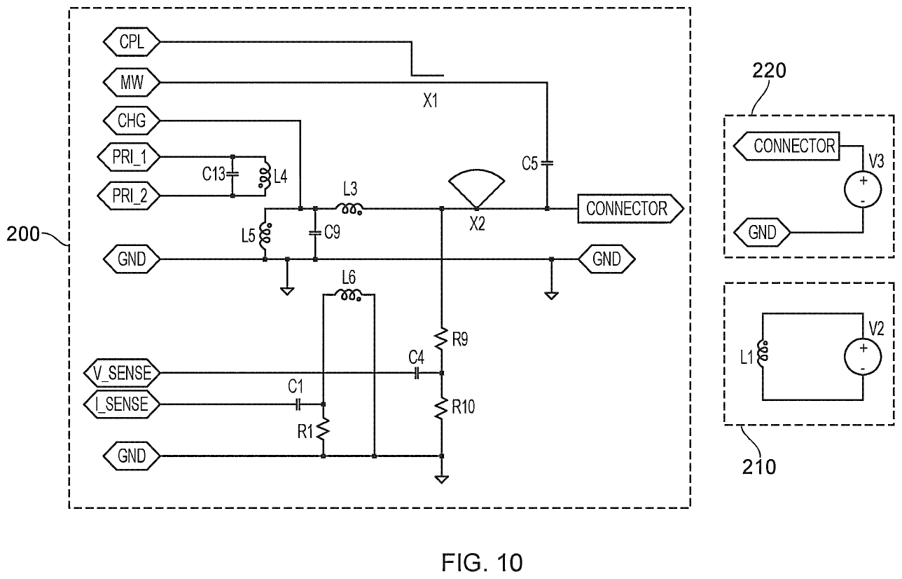

FIG. 10 shows a circuit diagram of an electrosurgical system including: an electrosurgical apparatus 200, a transmitter 210, and a wired charger 220. In this embodiment, the electrosurgical apparatus 200 contains a receiver circuit which is configured to allow both wireless and wired charging of the rechargeable power source. Other components may be included in addition to those shown, according to requirements. It is to be understood that for clarity the circuit diagram of FIG. 10 is a generalised schematic of the output sections of the electrosurgical apparatus that relate to wireless and wired charging. Remaining aspects of the electrosurgical apparatus would be clear to the skilled person from the previous figures that are described above.

In this embodiment, the electrosurgical apparatus 200 comprises two oscillators. A first oscillator provides microwave frequency energy via a microwave channel and the input MW (the input MW may form part of the microwave channel). A second oscillator provides RF energy via an RF channel and inputs PRI_1, PRI_2 (the inputs PRI_1 and PRI_2 may form part of the RF channel). The RF channel comprises a transformer having a primary coil (L4) and a secondary coil

(L5), which function in a similar manner as described with respect to FIGS. 3, 4 and 6 above. The RF channel also comprises capacitors (C9, C13) connected in parallel on either side of the transformer. The capacitors (C9, C13) help form a filter structure with the transformer (L4, L5) to improve conveyance of RF power to the output connector (CONNECTOR, GND) and also block unwanted harmonics of the RF power that in some circumstances may cause electromagnetic interference.

The microwave channel and the RF channel are each connected to the output (CONNECTOR, GND) in order to supply microwave and/or RF energy to an electrosurgical instrument, for example. In some embodiments the output (CONNECTOR, GND) may comprise a QMA connector or the like. A choke (X2) and a capacitor (C5) form an example of a combiner circuit which allows energy from both the microwave channel and the RF channel to reach the output (CONNECTOR, GND) while also preventing microwave energy reaching the RF channel and RF energy reaching the microwave channel. For example, the choke (X2) may be a quarter-wave short-circuit which may be implemented as a microstrip, a stripline, or a cavity resonator.

It is to be understood that the RF channel and the microwave channel may include one or more additional components, as described above with reference to FIG. 1.

Although a controller is not directly shown, sensing circuitry is indicated (CPL, V_SENSE, I_SENSE, GND) which is connected to a controller to allow the controller to monitor the RF or microwave energy which is delivered and/or reflected. A coupler (X1) is present on the microwave channel to allow the controller to sense the microwave power (CPL); the coupler (X1) is not sensitive to RF power. A capacitor (C5) ensures that RF power is prevented from reaching the microwave oscillator and the coupler (X1) due to its high impedance. A RF current-sensing circuit is formed by a transformer having a primary winding (L3) and a secondary winding (L6), a resistor (R1) and, optionally, a DC blocking capacitor (C1)—the RF current-sensing circuit is used to sense a proportion of the RF current flowing to the connector (CONNECTOR, GND) and is not sensitive to microwave power. A RF voltage-sensing circuit is formed by a potential divider connected to the RF channel and comprising two resistors (R9, R10) and, optionally, a DC blocking capacitor (C4)—the RF voltage-sensing circuit measures a proportion of the RF output voltage. The RF current-sensing circuit (L3, L6, R1, C1), RF voltage-sensing circuit (R9, R10, C4) and the microwave power sensing coupler (X1) are not essential to the operation of the charging system (either wired or wireless charging) and are shown only as an example to demonstrate how the circuit may be arranged to allow the control to monitor RF and/or microwave delivery.

The electrosurgical apparatus 200 also comprises a receiver circuit, which is connected to a rechargeable power source (not shown) via connection CHG. The receiver circuit is configured to allow charging by means of a wired or a wireless connection. The receiver circuit includes the secondary coil (L5) which forms an inductive coupler for wirelessly receiving power from the transmitter 210. The receiver circuit also includes the output (CONNECTOR, GND) for receiving power via a wired connection from the wired charger 220. It is to be understood that the receiver circuit may include one or more additional components as described above with reference to the previous figures.

A transmitter 210 comprises a power source (V2) and a transmitting inductive coupler (L1), which may be used to induce a current in the secondary coil (L5) of the transformer on the RF channel to allow wireless charging in substantially the manner described above with respect to FIGS. 3, 4, 6 and 7 above. In some embodiments, the transmitter 210 may additionally comprise a capacitor and, optionally, a resistor to allow wireless charging by resonant inductive coupling. The current induced in the secondary coil (L5) of the transformer is prevented from reaching the microwave channel by the capacitor (C5).

A wired charger 220 comprises a power source (V3) and a pair of contacts (CONNECTOR, GND). The power source (V3) may be mains power, for example, or may be a power source (e.g. a battery) internal to the wired charger 220. The wired charger 220 is configured to deliver energy into the electrosurgical apparatus 200 and to the receiver circuit via a connector formed by the output (CONNECTOR, GND). In other embodiments, the electrosurgical apparatus 200 may comprise one or more additional contacts which are configured to couple with the wired charger 220 to deliver energy to the receiver circuit. The current provided from the wired charger (220) is prevented from reaching the microwave channel by the capacitor (C5).

It is to be understood that in one version of FIG. 10, the transmitter 210 and the wired charger 220 are physically separate devices. For example, the transmitter 210 may be a wireless charging cradle similar to the one shown in FIG. 8a, and the wired charger may be a separate connecting device (e.g. cable) for connecting to a mains supply. However, in another version of FIG. 10, the transmitter 210 and the wired charger 220 may be housed within the same physical device. For example, the device may be a charging cradle similar to the one shown in FIG. 8a, but modified in order to provide wired charging from a power source (e.g. battery) contained within the cradle.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. An electrosurgical apparatus comprising:
an oscillator for generating electromagnetic energy;
a controller operable to select an energy delivery profile for the oscillator;
a feed structure for conveying the electromagnetic energy to an output;
a rechargeable power source arranged to supply power to the oscillator; and
a receiver circuit comprising an inductive coupler configured to wirelessly receive power from a transmitter and supply received power to the rechargeable power source, wherein
the feed structure comprises a transformer, and
wherein the inductive coupler comprises a secondary coil of the transformer.

2. (canceled)

3. (canceled)

2. An electrosurgical apparatus according to claim 1, wherein for every one turn of a primary coil of the transformer there are at least ten turns of a secondary coil of the transformer.

3. An electrosurgical apparatus according to claim 1, wherein the apparatus comprises a radiofrequency electromagnetic energy generator, and wherein the feed structure comprises a radiofrequency channel to convey the radiofrequency electromagnetic energy to the output.

4. An electrosurgical apparatus according to claim 1, wherein the apparatus comprises a microwave frequency electromagnetic energy generator, and wherein the feed structure comprises a microwave channel to convey the microwave frequency electromagnetic energy to the output.

5. An electrosurgical apparatus according to claim 1, wherein the rechargeable power source is a lithium-ion polymer battery.

6. An electrosurgical apparatus according to claim 1, further comprising a switching circuit operable to switch the rechargeable power source between a first mode for receiving power from the receiver circuit and a second mode for providing power to the oscillator.

7. An electrosurgical apparatus according to claim 6, wherein the controller is configured to operate the switching circuit.

8. An electrosurgical apparatus according to claim 1, wherein the receiver circuit is configured to allow wired charging of the rechargeable power source.

9. An electrosurgical apparatus according to claim 8, wherein the output forms a connector configured to receive energy for charging the rechargeable power source.

10. An electrosurgical apparatus according to claim 1, further comprising an electrosurgical instrument connected to receive electromagnetic energy from the output.

11. An electrosurgical apparatus according to claim 10, wherein the electrosurgical instrument is detachably connected to the output.

12. An electrosurgical apparatus according to claim 10 or claim 11, wherein the electro surgical instrument is a bipolar coaxial cutting tool.

13. An electrosurgical apparatus according to claim 1, wherein the electrosurgical apparatus comprises a housing which is adapted to be handheld by a user.

14. An electrosurgical system comprising:
an electrosurgical apparatus according to any one of claim 1; and
a transmitter for wirelessly providing power to the electrosurgical apparatus.

15. An electrosurgical system according to claim 14, wherein the transmitter comprises a transmitter circuit having an inductive coupler arranged to transmit power to the receiver circuit via inductive coupling.

16. An electrosurgical system according to claim 14 or, wherein the transmitter comprises a housing which is adapted to receive a portion of the electrosurgical apparatus.

17. An electrosurgical system according claim 14, further comprising a wired charger configured to form a wired electrical connection with the electrosurgical apparatus for providing wired power transfer to the electrosurgical apparatus.
